(19) United States
(12) Patent Application Publication
(10) Pub. No.: US 2017/0105959 A1
(43) Pub. Date: Apr. 20, 2017

(54) COMPOSITIONS CONTAINING NITRIC OXIDE AMINO ACID ESTERS AND METHODS FOR IMPROVING SEXUAL PERFORMANCE

(71) Applicant: Michael Farber, Livingston, NJ (US)

(72) Inventor: Michael Farber, Livingston, NJ (US)

(21) Appl. No.: 14/885,332

(22) Filed: Oct. 16, 2015

(51) Int. Cl. A61K 31/223; A61K 31/405; A61K 9/127; A61K 9/00; A61K 36/185; A61K 31/4172; A61K 31/225; A61K 36/74; A61K 31/475; A61K 38/05; A61K 31/216; A61K 31/401

(52) U.S. Cl. CPC A61K 31/223; A61K 31/216; A61K 31/405; A61K 9/127; A61K 9/006; A61K 31/401; A61K 31/4172; A61K 31/225; A61K 36/74; A61K 31/475; A61K 38/05; A61K 36/185

(57) ABSTRACT

Described herein are compositions and methods for enhancing or improving sexual performance or for treating sexual dysfunction or impotence comprising administering an effective amount of:
a compound of formula (I):

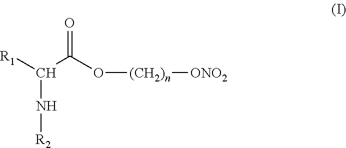

wherein n=1 to 10;
wherein R₁ is an amino acid side chain group (D or L configuration), or derivatives thereof,
wherein R₂ is a hydrogen atom, or an amino acid (D or L configuration) and derivatives thereof, forming a peptide bond;
or any pharmaceutically acceptable salts thereof; and
at least one a nutraceutical effective to enhance sexual performance
   in association with a pharmaceutically acceptable carrier.

### FIELD OF THE INVENTION

The present invention relates to compositions and methods for improving sexual performance or treating sexual dysfunction or impotence, and more particularly to compositions and methods for comprising a nitric oxide amino acid ester compound and a nutraceutical effective to enhance sexual performance.

### BACKGROUND OF THE INVENTION

Nitric oxide (NO) is synthesized from L-Arginine through the action of the enzyme NOs (nitric oxide synthetase). NO is synthesized by different NOs enzymes: nNOs (neuronal NOs), which is present in the cytoplasm of the parasympathetic nerves, and eNOs (endothelial NOs), found in the endothelium of the blood vessels and trabecular tissue, which mainly seems to bond to the cell membranes. Numerous experiments have demonstrated that stimulation of the parasympathetic nerves leads to the release of NO as a result of direct action by the nerve endings (reaction catalyzed by nNOs) and indirect action resulting from the effect of Ach, released by the parasympathetic nerves, on the vascular endothelium, with stimulation of eNos.

It is thus desirable to provide a composition and method for the which contains a Nitric Oxide releasing compound, and one or more nutraceutical effective to enhance sexual performance.

Sexual dysfunctions, particularly impotence, affect a substantial number of patients. Sexual dysfunction may include disorders of the cavernous tissue of the penis and the associated fascia, which produce impotence, the inability to attain a sexually functional erection. Impotence is estimated to affect approximately 30 million American men and can result from any of numerous physiological or psychological factors that may cause blood flow to and from the penis to remain in balance and preventing retention of sufficient blood to cause rigid dilation of the corpus cavernosa and spongiosa.

Erection takes place when the smooth muscles of the corpora cavernosa relax in response to sexual stimulation of the penis allowing the vessels to become engorged with blood. Sexual dysfunction or the inability to obtain an erection may result from a defect in neurovascular pathways that produce erection. Female sexual dysfunction is a significant age-related, progressive and highly prevalent problem affecting women in the United States.

The female sexual response is initiated by neurotransmitter-mediated vascular and nonvascular smooth muscle relaxation resulting in increased pelvic blood flow, vaginal lubrication, and clitoral and labial engorgement. Physiological impairments that interfere with the normal female sexual response bring about diminished sexual arousal, libido, vaginal lubrication, genital sensation, and ability to achieve orgasm. The causes of impotence in men may be multi-factorial, including psychopathology, diabetic neuropathy, aging and arteriosclerosis. Treatments for impotence include psychosexual therapy, oral sildenafil (Viagra®, Pfizer), intracavernosal injections or transurethral administration of the prostaglandin alprostadil, external vacuum devices, and implanted penile prostheses. Implants, requiring surgical insertion, are reserved for those who cannot be adequately treated by the less invasive measures. Ralph and McNicholas, 2000; Brit Med J. 321:499-503. Even the non-invasive treatments have disadvantages as well as advantages.

Psychosexual therapy may be used in conjunction with physical therapies. Sublingual apomorphine has been used to produce erection via stimulation of the mid-brain dopamine receptors. Oral sildenafil, (Viagra®), has been an effective treatment for erectile dysfunction. Side effects are predominantly mild. It is contraindicated in patients taking nitrates or with severe hepatic impairment, hypotension, hereditary degenerative retinal disorders, and recent stroke or myocardial infarction. Intracavernosal prostaglandin (alprostadil) injections are available that offer different injection systems to administer via the intracavernosal route. Alprostadil may also be given transurethrally in systems developed by Vivus, Inc. of Menlo Park, Calif. that have been proven effective in providing erections adequate for intercourse. Various other devices and compounds may be used for transurethral administration. e(U.S. Pat. Nos. 6,037,346, and 6,127,363) including phosphodiesterase inhibitors. Prostaglandin E has also been used topically to promote female sexual response. (U.S. Pat. No. 5,891,915)

Intracavernosal administration of phosphodiesterase type 5 inhibitors, such as sildenafil, or short-acting alpha-adrenergic receptor antagonists, such as phentolamine, in the absence of sexual stimulation do not initiate penile erection. In contrast, intracavernosal administration of Prostaglandin E1 or papaverine induces erection independent of sexual stimulation. Oral agents are not direct mediators of smooth muscle relaxation and act to facilitate relaxation in response to sexual stimulation, while intracavernosal agents directly mediate smooth muscle relaxation, independent of sexual stimulation. (Lue et al., Intl. J. Impotence Res. 12(S1):81-88 (2000)) Intracavernous injection of vasoactive drugs can produce a relatively rapid onset of erection in patients suffering from impotence due to venous leakage or arterial insufficiency. (Althof et al., J. Sex & Marital Therapy 5(2):121-29 (1989)). Other agents have been used topically, by intracavernosal injection, transurethral, topical and dermal administration. Piperoxan has been used by intracavernosal or transurethral injection. (U.S. Pat. No. 5,583,144) Mononitrates or dinitrates have been used by topical routes. (U.S. Pat. No. 6,056,966)

Natural and holistic compositions are also used to restore potency. A a mixture of lyophilized roe and a dry powdered extract from leaves of Ginkgo biloba (U.S. Pat. No. 5,730,987); a combination of the edible Andean tuber (Lepidium meyenii), known as maca, with deer or elk antler (U.S. Pat. No. 6,093,421); L-arginine, ginseng and Zizyphi fructus with or without Saw Palmetto, Gingko biloba, Glutamic acid and L-lysine (U.S. Pat. No. 6,007,824); a combination of Muira pauma, Yohimbe, Epimedium, L-arginine and Tribulus terrestris (Erect-X by Amassed Data of Los Angeles, Calif.); milled seeds from Aframomum stipulatum (U.S. Pat. No. 5,879,682).

A sexual treatment should be easy and simple to use and rapidly effective. Nutritional supplements and pharmaceutical agents are typically provided in solid dosage formulations that are taken orally including coated tablets, compressed tablets, compressed capsules and two piece gelatin capsules. These forms are easy and relatively inexpensive to produce, readily dispensable, and fairly stable. Solid dosage formulations of drugs and nutritional supplements have the disadvantage that stomach acids degrade many of the ingredients in the supplement. Degradation can be an important factor that limits the effectiveness of drugs and nutritional supplements, such as plant extracts, which are taken for medicinal purposes. Some biologically active compounds are delivered sublingually to achieve a rapid onset and greater bioavailability. By administering nitroglycerin tablets under the tongue, rapid onset is achieved by virtue of quick absorpttion into the blood stream through the highly vascularized capillary plexus. In addition, this route avoids liver metabolismon first exposure

The mucosa of the mouth and throat is highly vascularized and well suited for the absorption of lipophilic, nonionized compounds.

The major types of liposomes are the multilamellar vesicle (MLV, with several lamellar phase lipid bilayers), the small unilamellar liposome vesicle (SUV, with one lipid bilayer), the large unilamellar vesicle (LUV), and the cochleate vesicle. A less desirable form are multivesicular liposomes in which one vesicle contains one or more smaller vesicles.

A liposome has an aqueous solution core surrounded by a hydrophobic membrane, in the form of a lipid bilayer; hydrophilic solutes dissolved in the core cannot readily pass through the bilayer. Hydrophobic chemicals associate with the bilayer. A liposome can be hence loaded with hydrophobic and/or and hydrophilic molecules. To deliver the molecules to a site of action, the lipid bilayer can fuse with other bilayers such as the cell membrane, thus delivering the liposome contents; this is a complex and non-spontaneous event, however. Cevc, Advanced Drug Delivery Reviews, 1993; 38 (3): 207-232 By preparing liposomes in a solution of DNA or drugs (which would normally be unable to diffuse through the membrane) they can be (indiscriminately) delivered past the lipid bilayer, but are then typically distributed non-homogeneously. Barenholz, et al., (2000). Physical chemistry of biological surfaces, Chapter 7: Structure and properties of membranes. New York: Marcel Dekker. pp. 171-241.

Liposomes are used as models for artificial cells. Liposomes can also be designed to deliver drugs in other ways. Liposomes that contain low (or high) pH can be constructed such that dissolved aqueous drugs will be charged in solution (i.e., the pH is outside the drug's pI range). As the pH naturally neutralizes within the liposome (protons can pass through some membranes), the drug will also be neutralized, allowing it to freely pass through a membrane. These liposomes work to deliver drug by diffusion rather than by direct cell fusion.

A similar approach can be exploited in the biodetoxification of drugs by injecting empty liposomes with a transmembrane pH gradient. In this case the vesicles act as sinks to scavenge the drug in the blood circulation and prevent its toxic effect. Bertrand, et al., ACS Nano 4 2000; (12): 7552-8 Another strategy for liposome drug delivery is to target endocytosis events. Liposomes can be made in a particular size range that makes them viable targets for natural macrophage phagocytosis. These liposomes may be digested while in the macrophage's phagosome, thus releasing its drug. Liposomes can also be decorated with opsonins and ligands to activate endocytosis in other cell types. The use of liposomes for transformation or transfection of DNA into a host cell is known as lipofection.

As of 2012, some 13 drugs with liposomal delivery systems have been approved and five additional liposomal drugs were in clinical trials. The clinically approved liposomal drugs include amphotericin B, ctyarabine, daunorubicin, doxorubicin, IRW vaccine, morphine, verteporfin, proteins SP-B and SP-C, estradiol, vincristine, and PEG.

Liposomes rarely form spontaneously. They typically form after supplying enough energy to a dispersion of (phospho)lipids in a polar solvent, such as water, to break down multilamellar aggregates into oligo- or unilamellar bilayer vesicles. Cevc, Journal of Controlled Release, 1993; 160 (2): 135-146; Barenholz, et al., (2000). Physical chemistry of biological surfaces, Chapter 7: Structure and properties of membranes. New York: Marcel Dekker. pp. 171-241.

Liposomes can hence be created by sonicating a dispersion of amphipatic lipids, such as phospholipids, in water. Low shear rates create multilamellar liposomes. The original aggregates, which have many layers like an onion, thereby form progressively smaller and finally unilamellar liposomes (which are often unstable, owing to their small size and the sonication-created defects). Sonication is generally considered a “gross” method of preparation as it can damage the structure of the drug to be encapsulated. Newer methods such as extrusion and Mozafari method are employed to produce materials for human use. Colas, et al., “Microscopical investigations of nisin-loaded nanoliposomes prepared by Mozafari method and their bacterial targeting”. Micron (Oxford, England: 1993) 2007; 38 (8): 841-7 Using lipids other than phosphatidylcholine can greatly facilitate liposome preparation. Cevc, Journal of Controlled Release, 1993; 160 (2):135-146.

### SUMMARY OF THE INVENTION

In a first aspect, the invention features compositions for enhancing or improving sexual performance or treating sexual dysfunction or impotence featuring
an effective amount of a compound of formula (I):

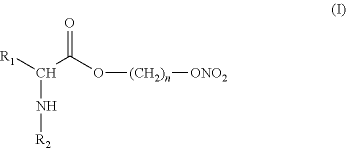

wherein n=1 to 10;
wherein R₁ is chosen an amino acid side chain group (D or L configuration), or derivatives thereof,
wherein R₂ is a hydrogen atom, or an amino acid (D or L configuration) and derivatives thereof, forming a peptide bond, or any pharmaceutically acceptable salts thereof; and
an effective amount of a nutraceutical effective to enhance sexual performance, in association with a pharmaceutically acceptable topical carrier.

In preferred embodiments, n=2.

The compound of formula (I) may be (2-nitrooxy)-2-ethylamino-3-methylbutanoate:

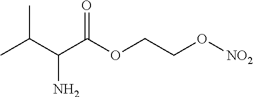

or any pharmaceutically acceptable salts thereof.

The compound of formula (I) may be valine butylene glycol nitrate:

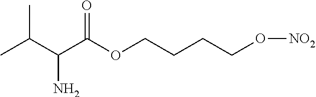 [SMILES: CC(C)C(N)C(=O)OCCCCO[N+](=O)[O-]]

or any pharmaceutically acceptable salts thereof.

The compound of formula (I) may be 2′-nitrooxy ethyl 2-amino-pentanoate:

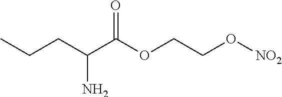

or any pharmaceutically acceptable salts thereof.

The compound of formula (I) may be 2′-nitrooxy butyl 2-amino-pentanoate:

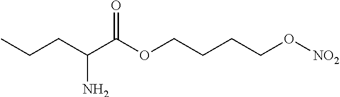 [SMILES: CCCC(N)C(=O)OCCCCO[N+](=O)[O-]]

or any pharmaceutically acceptable salts thereof.

R₁ may be selected from the group consisting of:

| Originating | | |
|---|---|---|
| Amino acid | Formula | R₁ |
| Glycine | H | H |
| Alanine | CH₃ | CH₃ |
| Valine | (CH₃)₂ | 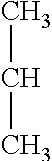 |
| Leucine | CH₂CH(CH₃)₂ | 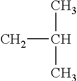 |
| Isoleucine | CH(CH₃)CH₂CH₃ | 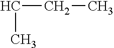 |
| Phenylalanine | CH₂C₆H₅ | 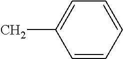 |
| Tyrosine | CH₂C₆H₄OH | 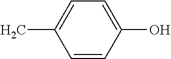 |
| Tryptophane | C₉H₈N | 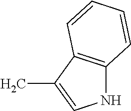 |
| Serine | CH₂OH | H₂C—OH |
| Threonine | CH(OH)CH₃ | 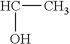 |
| Cysteine | CH₂SH | H₂C—SH |
| Methionine | CH₂CH₂SCH₃ | 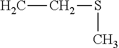 |
| Proline | C₅H₉NO₂ | 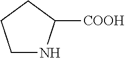 |
| Asparagine | CH₂COCH₂ | 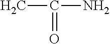 |
| Glutamine | CH₂CH₂CONH₂ | 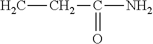 |
| Aspartic acid | CH₂COOH | CH₂—COOH |
| Glutamic acid | CH₂CH₂COOH | H₂C—CH₂—COOH |
| Lysine | CH₂CH₂CH₂CH₂NH₂ | 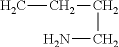 |
| Histidine | CH₃C₃N₂H₃ | 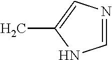 |
| Arginine | (CH₂)₃CN₃H₄ | 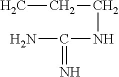 |
| Cystine | CH₂S₂CH₂CHNH₂COOH | 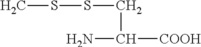 |
| Hydroxyproline | | 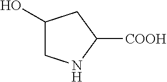 |
| ε-N- methyllysine | CH₂CH₂CH₂CH₂NHCH₃ | 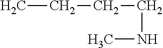 |
| β-alanine | NH₂CH₂CH₂COOH | 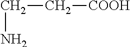 |
| diiodotyrosine | CH₂C₆H₂I₂OH | 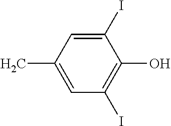 |
| homocysteine | CH₂CH₂SH | H₂C—CH₂—SH |
| ornithine | CH₂CH₂CH₂NH₂ | 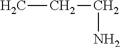 |
| Norvaline | CH₂—CH₃ | CH₂—CH₃ |
| selenocysteine | SeH | SeH |
| Hypusine | CH₂CH₂CH₂CH₂NHCH₂CH(OH)CH₂CH₂NH₂ | 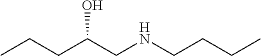 |
| Dehydroalanine | CH₂ | 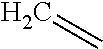 |

R₂ is an amino acid of formula (II) (D or L configuration) and derivatives thereof, forming a peptide bond:

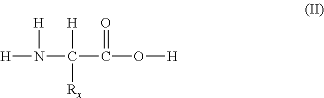

wherein Rₓ is chosen from

| Originating | | |
|---|---|---|
| Amino acid | Formula | Rₓ |
| Glycine | H | H |
| Alanine | CH₃ | CH₃ |
| Valine | (CH₃)₂ | 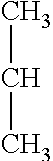 |
| Leucine | CH₂CH(CH₃)₂ | 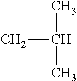 |
| Isoleucine | CH(CH₃)CH₂CH₃ | 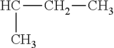 |
| Phenylalanine | CH₂C₆H₅ | 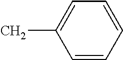 |
| Tyrosine | CH₂C₆H₄OH | 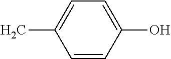 |
| Tryptophane | C₉H₈N | 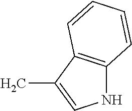 |
| Serine | CH₂OH | H₂C—OH |
| Threonine | CH(OH)CH₃ | 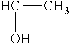 |
| Cysteine | CH₂SH | H₂C—SH |
| Methionine | CH₂CH₂SCH₃ | 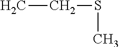 |
| Proline | C₅H₉NO₂ | 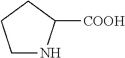 |
| Asparagine | CH₂COCH₂ | 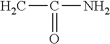 |
| Glutamine | CH₂CH₂CONH₂ | 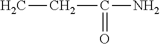 |
| Aspartic acid | CH₂COOH | CH₂—COOH |
| Glutamic acid | CH₂CH₂COOH | H₂C—CH₂—COOH |
| Lysine | CH₂CH₂CH₂CH₂NH₂ | 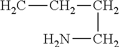 |
| Histidine | CH₃C₃N₂H₃ | 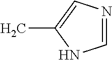 |
| Arginine | (CH₂)₃CN₃H₄ | 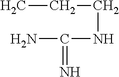 |
| Cystine | CH₂S₂CH₂CHNH₂COOH | 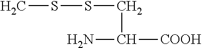 |
| Hydroxyproline | | 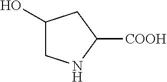 |
| ε-N-methyllysine | CH₂CH₂CH₂CH₂NHCH₃ | 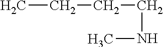 |
| β-alanine | NH₂CH₂CH₂COOH | 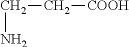 |
| diiodotyrosine | CH₂C₆H₂I₂OH | 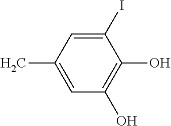 |
| homocysteine | CH₂CH₂SH | H₂C—CH₂—SH |
| ornithine | CH₂CH₂CH₂NH₂ | 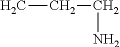 |
| Norvaline | CH₂—CH₃ | CH₂—CH₃ |
| selenocysteine | SeH | SeH |
| Hypusine | CH₂CH₂CH₂CH₂NHCH₂CH(OH)CH₂CH₂NH₂ | 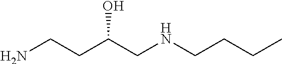 |
| Dehydroalanine | CH₂ | 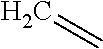 |

The nutraceutical effective to enhance sexual performance may be Pausinystalia yohimbe, yohimbine, alpha-yohimbine, muira puama, ginseng extract, or Damiana extract. The nutraceutical effective to enhance sexual performance may be liposome encapsulated. The liposome-encapsulated nutraceutical effective to enhance sexual performance such as liposome encapsulated Pausinystalia yohimbe, yohimbine, alpha-yohimbine, muira puama, ginseng extract, or Damiana extract may be multilamellar, unilamellar, or multivesicular lipid vesicles or a multiphase liposomal system and may be prepared using a phospholipid such as, for instance, phosphatidylcholines, lysophosphatidylcholines, phosphatidylserines, phosphatidylethanolamines, phosphatidylinositols and mixtures thereof. Preferably, the phospholipid is natural soybean lecithin. The phospholipid may also be provided in mixtures with a cholesterol, stearyl amine, stearic acid, or tocopherol.

The nutraceutical effective to enhance sexual performance such as liposome encapsulated Pausinystalia yohimbe, yohimbine, alpha-yohimbine, muira puama, ginseng extract, or Damiana extract may be present in amounts sufficient to treat sexual dysfunction or improve sexual function.

The compound of Formula I may be present in an amount of 5 to 50 mg, preferably 10 to 30 mg. The nutraceutical effective to enhance sexual performance may be present in an amount of 5 to 25 mg, 5 to 10 mg where the nutraceutical is yohimbine extract 98%, and the muira puama may be preferably a 40:1 ethanolic extract. The composition may be therapeutically or clinically effective within about 5, 10, 15, 20, 30, 45 minutes or 1 or 2 hours.

In a second embodiment, the present invention provides a method for enhancing or improving sexual performance or treating sexual dysfunction or impotence in a patient comprising:

administering a therapeutically effective amount of a composition comprising
an effective amount of a compound of formula (I):

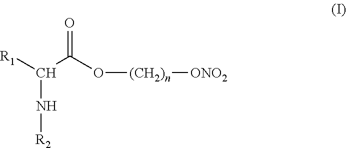

wherein n=1 to 10;
wherein R₁ is chosen an amino acid side chain group (D or L configuration), or derivatives thereof,
wherein R₂ is a hydrogen atom, or an amino acid (D or L configuration) and derivatives thereof, forming a peptide bond, or any pharmaceutically acceptable salts thereof; and
an effective amount of a nutraceutical effective to enhance sexual performance.

Preferably n=2. The composition may be administered orally or sublingually, and it may be administered in tablet form. Preferably, the nutraceutical effective to enhance sexual performance is encapsulated in a liposome.

The compound of formula (I) may be (2-nitrooxy)-2-ethylamino-3-methylbutanoate:

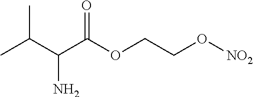

or any pharmaceutically acceptable salts thereof.

The compound of formula (I) may be valine butylene glycol nitrate:

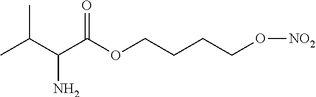 [SMILES: CC(C)C(N)C(=O)OCCCCO[N+](=O)[O-]]

or any pharmaceutically acceptable salts thereof.

The compound of formula (I) may be 2′-nitrooxy ethyl 2-amino-pentanoate:

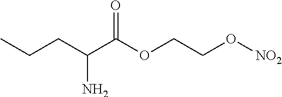

or any pharmaceutically acceptable salts thereof.

The compound of formula (I) may be 2′-nitrooxy butyl 2-amino-pentanoate:

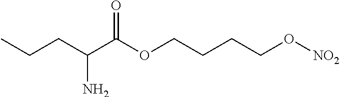 [SMILES: CCCC(N)C(=O)OCCCCO[N+](=O)[O-]]

or any pharmaceutically acceptable salts thereof.

R₁ may be selected from the group consisting of:

| Originating | | |
|---|---|---|
| Amino acid | Formula | R₁ |
| Glycine | H | H |
| Alanine | CH₃ | CH₃ |
| Valine | (CH₃)₂ | 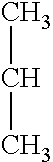 |
| Leucine | CH₂CH(CH₃)₂ | 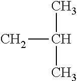 |
| Isoleucine | CH(CH₃)CH₂CH₃ | 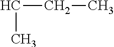 |
| Phenylalanine | CH₂C₆H₅ | 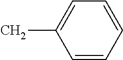 |
| Tyrosine | CH₂C₆H₄OH | 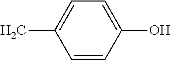 |
| Tryptophane | C₉H₈N | 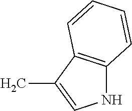 |
| Serine | CH₂OH | H₂C—OH |
| Threonine | CH(OH)CH₃ | 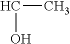 |
| Cysteine | CH₂SH | H₂C—SH |
| Methionine | CH₂CH₂SCH₃ | 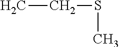 |
| Proline | C₅H₉NO₂ | 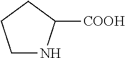 |
| Asparagine | CH₂COCH₂ | 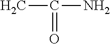 |
| Glutamine | CH₂CH₂CONH₂ | 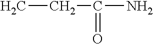 |
| Aspartic acid | CH₂COOH | CH₂—COOH |
| Glutamic acid | CH₂CH₂COOH | H₂C—CH₂—COOH |
| Lysine | CH₂CH₂CH₂CH₂NH₂ | 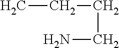 |
| Histidine | CH₃C₃N₂H₃ | 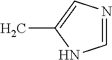 |
| Arginine | (CH₂)₃CN₃H₄ | 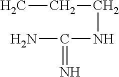 |
| Cystine | CH₂S₂CH₂CHNH₂COOH | 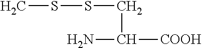 |
| Hydroxyproline | | 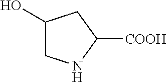 |
| ε-N-methyllysine | CH₂CH₂CH₂CH₂NHCH₃ | 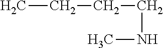 |
| β-alanine | NH₂CH₂CH₂COOH | 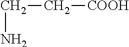 |
| diiodotyrosine | CH₂C₆H₂I₂OH | 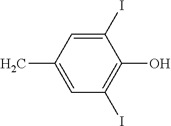 |
| homocysteine | CH₂CH₂SH | H₂C—CH₂—SH |
| ornithine | CH₂CH₂CH₂NH₂ | 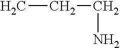 |
| Norvaline | CH₂—CH₃ | CH₂—CH₃ |
| selenocysteine | SeH | SeH |
| Hypusine | CH₂CH₂CH₂CH₂NHCH₂CH(OH)CH₂CH₂NH₂ | 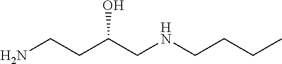 |
| Dehydroalanine | CH₂ | 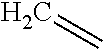 |

R₂ is an amino acid of formula (II) (D or L configuration) and derivatives thereof, forming a peptide bond:

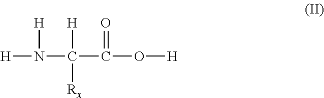

wherein Rₓ is chosen from

| Originating | | |
|---|---|---|
| Amino acid | Formula | Rₓ |
| Glycine | H | H |
| Alanine | CH₃ | CH₃ |
| Valine | (CH₃)₂ | 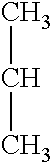 |
| Leucine | CH₂CH(CH₃)₂ | 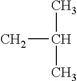 |
| Isoleucine | CH(CH₃)CH₂CH₃ | 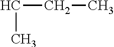 |
| Phenylalanine | CH₂C₆H₅ | 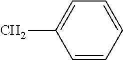 |
| Tyrosine | CH₂C₆H₄OH | 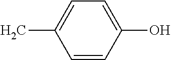 |
| Tryptophane | C₉H₈N | 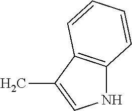 |
| Serine | CH₂OH | H₂C—OH |
| Threonine | CH(OH)CH₃ | 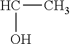 |
| Cysteine | CH₂SH | H₂C—SH |
| Methionine | CH₂CH₂SCH₃ | 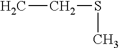 |
| Proline | C₅H₉NO₂ | 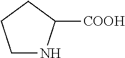 |
| Asparagine | CH₂COCH₂ | 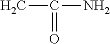 |
| Glutamine | CH₂CH₂CONH₂ | 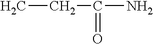 |
| Aspartic acid | CH₂COOH | CH₂—COOH |
| Glutamic acid | CH₂CH₂COOH | H₂C—CH₂—COOH |
| Lysine | CH₂CH₂CH₂CH₂NH₂ | 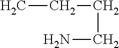 |
| Histidine | CH₃C₃N₂H₃ | 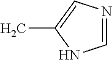 |
| Arginine | (CH₂)₃CN₃H₄ | 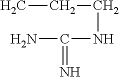 |
| Cystine | CH₂S₂CH₂CHNH₂COOH | 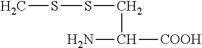 |
| Hydroxyproline | | 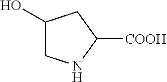 |
| ε-N-methyllysine | CH₂CH₂CH₂CH₂NHCH₃ | 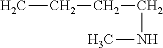 |
| β-alanine | NH₂CH₂CH₂COOH | 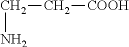 |
| diiodotyrosine | CH₂C₆H₂I₂OH | 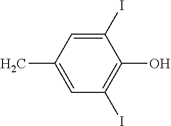 |
| homocysteine | CH₂CH₂SH | H₂C—CH₂—SH |
| ornithine | CH₂CH₂CH₂NH₂ | 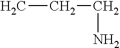 |
| Norvaline | CH₂—CH₃ | CH₂—CH₃ |
| selenocysteine | SeH | SeH |
| Hypusine | CH₂CH₂CH₂CH₂NHCH₂CH(OH)CH₂CH₂NH₂ | 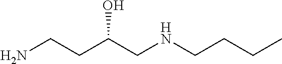 |
| Dehydroalanine | CH₂ | 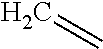 |

The nutraceutical effective to enhance sexual performance may be a liposome encapsulated Pausinystalia yohimbe, yohimbine, alpha-yohimbine, muira puama, ginseng extract, or Damiana extract. The liposome-encapsulated nutraceutical effective to enhance sexual performance such as liposome encapsulated Pausinystalia yohimbe, yohimbine, alpha-yohimbine, muira puama, ginseng extract, or Damiana extract may be multilamellar, unilamellar, or multivesicular lipid vesicles or a multiphase liposomal system and may be prepared using a phospholipid such as, for instance, phosphatidylcholines, lysophosphatidylcholines, phosphatidylserines, phosphatidylethanolamines, phosphatidylinositols and mixtures thereof. The phospholipid may be provided in admixtures with a cholesterol, stearyl amine, stearic acid, or tocopherol.

The nutraceutical effective to enhance sexual performance such as liposome encapsulated Pausinystalia yohimbe, yohimbine, alpha-yohimbine, muira puama, ginseng extract, or Damiana extract may be present in amounts sufficient to treat sexual dysfunction or improve sexual function.

The compound of Formula I may be present in an amount of 5 to 50 mg, preferably 10 to 30 mg. The nutraceutical effective to enhance sexual performance may be present in an amount of 5 to 25 mg, 5 to 10 mg where the nutraceutical is yohimbine extract 98%, and the muira puama is preferably a 40:1 ethanolic extract. The method may be therapeutically or clinically effective within about 5, 10, 15, 20, 30, 45 minutes or 1 or 2 hours.

### DETAILED DESCRIPTION OF THE PREFERRED EMBODIMENTS

The term “Amino acid ester compound” is intended to mean the condensation product of an amino acid with mononitrated alkane ou alkene diol. As will be evident to those familiar to the art, the condensation reaction could also involve, but not limited to, dipeptides or tripeptides, nitrated alcohols containing aliphatic, alkyl or aromatic moieties, as well as other nitric oxide groups attached to the alkane or alkene diols Amino acid or dipeptide reactions are preferred as well as the condensation reaction with short chain mononitrated alkane diols such as 1,3 propanediol or 1,4 butanediol.

The expression “Therapeutically effective amount” is intended to mean the amount of the compound and/or composition that is effective to achieve its intended purpose.

The term “Transmucosal” is intended to mean the delivery of a compound by passage of the compound through the mucosal tissue and into the blood stream.

The terms “Carriers” or “vehicles” are intended to mean carrier materials suitable for compound administration and include any such material known in the art such as, for example, any liquid, lotion, gel, solvent, liquid diluent, solubilizer, or the like, which is non-toxic and which does not interact with any components of the composition in a deleterious manner.

The term “Nitric oxide adduct” or “NO adduct” is intended to mean compounds and functional groups which, under physiological conditions, can donate, release and/or directly or indirectly transfer any of the three redox forms of nitrogen monoxide (NO⁺, NO⁻, NO*), such that the biological activity of the nitrogen monoxide species is expressed at the intended site of action.

The term “Nitric oxide releasing” or “nitric oxide donating” is intended to mean methods of donating, releasing and/or directly or indirectly transferring any of the three redox forms of nitrogen monoxide (NO+, NO−, NO*), such that the biological activity of the nitrogen monoxide species is expressed at the intended site of action.

The term “Nitric oxide donor” or “NO donor” is intended to mean compounds that donate, release and/or directly or indirectly transfer a nitrogen monoxide species, and/or stimulate the endogenous production of nitric oxide or endothelium-derived relaxing factor (EDRF) in vivo and/or elevate endogenous levels of nitric oxide or EDRF in vivo and/or are oxidized to produce nitric oxide and/or are substrates for nitric oxide synthase and/or cytochrome P450. “NO donor” also includes compounds that are precursors of L-arginine, inhibitors of the enzyme arginase and nitric oxide mediators.

The term “pharmaceutical acceptable carrier” is intended to mean a preservative solution, a saline solution, an isotonic (about 0.9%) saline solution, or about a 5% albumin solution, suspension, sterile water, phosphate buffered saline, and the like. Other buffering agents, dispersing agents, and inert non-toxic substances suitable for delivery to a patient may be included in the compositions of the present invention. The compositions may be solutions, suspensions or any appropriate formulation suitable for administration, and are typically sterile and free of undesirable particulate matter. The compositions may be sterilized by conventional sterilization techniques.

The term “excipient” refers to a natural or synthetic substance formulated alongside the active ingredient of a medication, included for the purpose of bulking up formulations that contain potent active ingredients (thus often referred to as “bulking agents,” “fillers,” or “diluents”), or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. The selection of appropriate excipients also depends upon the route of administration and the dosage form, as well as the active ingredient and other factors.

Features and advantages of the compositions and methods described herein will become more apparent in light of the following detailed description of selected embodiments. The compositions and methods described herein are capable of modifications in various respects, all without departing from the scope of the claims. Accordingly, the figures and the description are to be regarded as illustrative in nature, and not as restrictive and the full scope of the subject matter is set forth in the claims.

### Nitric Oxide Amino Acid Ester Compounds

The compositions of the present invention contain vasoactive amino acid ester compounds. The nitric oxide amino acid ester compounds of the present invention possess many of the required characteristics necessary to fulfill the role of a primary boosting of NO levels. The compounds easily dissociate in water into the amino acid derivative and associated ion forming the pharmaceutical salt. The compounds of the present invention are extremely stable in the form of the salts, and thus possess long shelf lives and stability.

The nitric oxide releasing groups of the compounds of the present invention are preferably nitro groups (i.e. NO₂), nitroso groups (i.e. NO) and/or heterocyclic nitric oxide donor groups that are linked to the amino acid ester compounds through one or more sites such as oxygen (hydroxyl condensation), sulfur (sulfhydryl condensation) and/or nitrogen. The heterocyclic nitric oxide donor groups are preferably furoxans, sydnonimines, oxatriazole-5-ones and/or oxatriazole-5-imines.

The preferred compound of the present invention is the valine derivative of the nitric oxide amino acid ester of the present invention. The most preferred compounds are known as valine nitrooxy ethyl ester (or valine ethylene glycol nitrate), valine nitrooxy butyl ester (or valine butylene glycol nitrate), or any pharmaceutically acceptable salts thereof, which possess many of the required characteristics necessary to fulfill the role of boosting NO levels. The compound easily dissociates in water into the valine derivative valine ethylene or butylene glycol nitrate and the salt forming acid. The compounds are extremely stable in the form of the salt and thus possesses a long shelf life. Another preferred compound is the norvaline derivative of the nitric oxide amino acid ester of the present invention. The most preferred compounds are known as norvaline nitrooxy ethyl ester (or norvaline ethylene glycol nitrate or 2′-nitrooxy ethyl 2-amino-pentanoate), norvaline nitrooxy butyl ester (or norvaline butylene glycol nitrate or 4′-nitrooxybutyl 2-amino-pentanoate), or any pharmaceutically acceptable salts thereof, which possess many of the required characteristics necessary to fulfill the role of boosting NO levels. The compound easily dissociates in water into the norvaline derivative norvaline ethylene or butylene glycol nitrate and the salt forming acid. The compounds are extremely stable in the form of the salt and thus possesses a long shelf life

The compounds and compositions of the invention can be formulated as pharmaceutically acceptable salt forms. Pharmaceutically acceptable salts include, for example, alkali metal salts and addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids include, but are not limited to, hydrochloric, hydrobromic, hydroiodic, nitric, carbonic, sulfuric and phosphoric acid and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, β-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N, N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound. In one embodiment, the pharmaceutically acceptable salts of the compounds of the invention include the nitrate salts. In another embodiment, the pharmaceutically acceptable salts of the compounds of the invention are heterocyclic compounds such as, furoxan, a sydnonimine, an oxatriazole-5-one and/or an oxatriazole-5-imine.

The compounds of the present invention, because of the small size of the molecule, can be other choices of linkages and/or amino acids or their derivatives. For example, as alternatives to the above choices, propyl, butyl, or longer chains may be linked to any amino acid. Salts such as chloride or hydrochloride salts may be used. Other amino acid derivatives may also be chosen. Derivatives of the base amino acids whether they are in the L or D configuration of these amino acids can be chosen. Non standard amino acids, or synthetic derivative of standard and non-standard amino acids may be elected, such as those containing acetyl groups attached to the amide of the molecule or nor derivatives of the amino acids, when such derivatives can be achieved.

The amino acid esters compounds may be based on natural, non-standard or even modified amino acids, with the basic structure as depicted below, where the Rₓ represents the side chain of the amino acid (wherein Rₓ may be R₁, R₂ or R₃, as applicable to the specific molecule described herein):

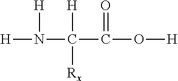

### Basic Amino Acid Structure

Natural Amino Acids

| | Originating Amino | | |
|---|---|---|---|
| No | acid | Formula | Rₓ = R₁ or R₂ or R₃ |
| 1 | Glycine | H | —H |
| 2 | Alanine | CH₃ | —CH₃ |
| 3 | Valine* | (CH₃)₂ | 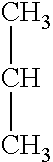 |
| 4 | Leucine* | CH₂CH(CH₃)₂ | 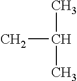 |
| 5 | Isoleucine* | CH(CH₃)CH₂CH₃ | 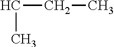 |
| 6 | Phenylalanine* | CH₂C₆H₅ | 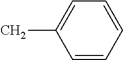 |
| 7 | Tyrosine | CH₂C₆H₄OH | 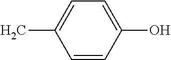 |
| 8 | Tryptophane* | C₉H₈N | 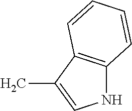 |
| 9 | Serine | CH₂OH | H₂C—OH |
| 10 | Threonine* | CH(OH)CH₃ | 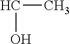 |
| 11 | Cysteine | CH₂SH | H₂C—SH |
| 12 | Methionine* | CH₂CH₂SCH₃ | 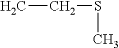 |
| 13 | Proline | C₅H₉NO₂ | 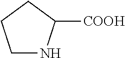 |
| 14 | Asparagine | CH₂COCH₂ | 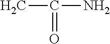 |
| 15 | Glutamine | CH₂CH₂CONH₂ | 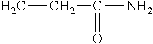 |
| 16 | Aspartic acid | CH₂COOH | CH₂—COOH |
| 17 | Glutamic acid | CH₂CH₂COOH | H₂C—CH₂—COOH |
| 18 | Lysine* | CH₂CH₂CH₂CH₂NH₂ | 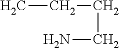 |
| 19 | Histidine* | CH₃C₃N₂H₃ | 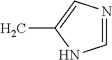 |
| 20 | Arginine* | (CH₂)₃CN₃H₄ | 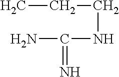 |

*essential amino acids

Modified Amino Acids

| | Originating | | |
|---|---|---|---|
| No | Amino acid | Formula | Rₓ = R₁ or R₂ or R₃ |
| A | Cystine | CH₂S₂CH₂CHNH₂COOH | 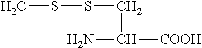 |
| B | Hydroxyproline | | 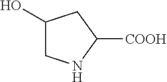 |
| C | ε-N-methyllysine | CH₂CH₂CH₂CH₂NHCH₃ | 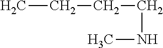 |
| D | β-alanine | NH₂CH₂CH₂COOH | 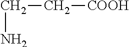 |
| E | diiodotyrosine | CH₂C₆H₂I₂OH | 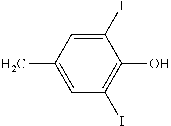 |
| F | homocysteine | CH₂CH₂SH | H₂C—CH₂—SH |
| G | ornithine | CH₂CH₂CH₂NH₂ | 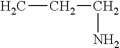 |
| H | Norvaline | CH₂—CH₃ | CH₂—CH₃ |
| I | selenocysteine | SeH | SeH |
| J | Hypusine | CH₂CH₂CH₂CH₂NHCH₂CH(OH)CH₂CH₂NH₂ | 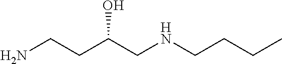 |
| K | Dehydroalanine | CH₂ | 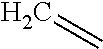 |

The nitric oxide amino acid ester compounds of the present invention are not limited to a single amino acid molecule. The compounds of the present invention may be dipeptide or even tripeptide molecules, with the general formula depicted below and where Rₓ and R_{y} independently are any of the amino acid side chains described herein.

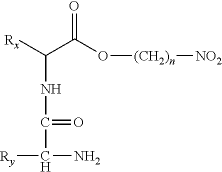

The composition containing a compound as defined in the present invention may include a wide variety of additional components, including, for example, one or more of gases, gaseous precursors, liquids, oils, stabilizing materials, pharmaceutical acceptable carriers, photoactive agents.

The invention provides methods for boosting NO levels for the treatment of sexual dysfunction or impotence or for improving sexual function.

### Liposomal Nutraceutical Compositions

The present invention also provides liposomal compositions containing a nutraceutical effective to enhance sexual performance and methods for treating sexual dysfunction or impotence or enhancing sexual function. The liposomes are preferably dispersed in a pharmaceutically acceptable carrier and contain yohimbine or a yohimbe extract or muira puama.

Yohimbine is an FDA approved medicine for impotence, an alkaloid isolated from the bark of the yohimbe tree (Pausinystalia yohimbe). It has long been used treat both male and female sexual difficulties. (Riley, Br. J. Clin. Pract. 1994; 48(3): 133-36). Yohimbine hydrochloride increases libido, but its primary action is to increase blood flow to erectile tissue. The engorgement of genital tissues in the corpora cavernosa of the male and the clitoris of the female occur via the same mechanisms.

Yohimbine or an extract of yohimbe is encapsulated in liposomes to increase its adsorption rate.

Ptychopetalum Olacoids (Muira puama)

Ptychopetalum olacoides has long been used as an aphrodisiac (Mowrey, Energytimes Magazine (February 1996)). It is an aphrodisiac herb indigenous to South America that stimulates male libido and overcome erectile dysfunction. It is considered to be one of the best agents to use for erectile dysfunction or lack of libido.

A recent study has validated its safety and effectiveness in improving libido and sexual function in some patients. At the Institute of Sexology in Paris, France, a clinical study with 262 patients complaining of lack of sexual desire and the inability to attain or maintain an erection demonstrated Muira puama extract to be effective in many cases. Within two weeks, at a daily dose of 1 to 1.5 grams of the extract (4:1), 62% of patients with loss of libido claimed that the treatment had dynamic effect while 51% of patients with “erection failures” felt that Muira puama was of benefit. (Ethnopharmacology, (Ed. Rivier L., & Anton, R., 1990); Waynberg, J., Proceedings of the First International Congress on Ethnopharmacology, Strasbourg, France, Jun. 5-9, 1990.)

Active constituents are free long-chain fatty acids, sterols, coumarin, alkaloids and essential oils. Chemically, Muira puama contains 0.05% muirapuamine, 0.4% fat, 0.5% alkaloids, 0.6% pholbaphene, 0.6% alpha-resinic acid, 0.7% beta resinic acid, 0.5% of a mixture of esters including behenic acid, lupeol and beta-sitosterol, as well as tannin, volatile oils and fatty acids. (CRC Handbook of Medicinal Herbs (Duke, J. 1985)). Proper preparation methods must be employed to preserve the active constituents found in the natural bark. The extract is produced by high heat for at least 20 minutes or longer in alcohol necessary to dissolve and extract the volatile and essential oils, terpenes, gums and resins found in the bark and root.

The administration of the composition is preferably by a predetermined regimen, which may be only on an as needed (“PRN”) basis, or at least once daily and over an extended period of time for chronic treatment, and could last for one year or more, including the life of the subject. The dosage administered will depend upon the frequency of the administration, the blood level desired, other concurrent therapeutic treatments, the severity of the condition, whether the treatment is for improving sexual function or treating sexual dysfunction.

Several methods for preparing liposomes are known in the art such as those described in U.S. Pat. No. 4,089,801, expressly incorporated herein by reference. A mixture of a lipid, an aqueous solution of the material to be encapsulated, and a liquid which is insoluble in water, is subjected to ultrasonication, whereby aqueous globules enclosed in a monomolecular lipid layer form liposome precursors. The lipid vesicles are then prepared by combining the first dispersion of liposome precursors with a second aqueous medium containing amphiphilic compounds, and subjecting the mixture to centrifugation, whereby the globules are forced through the monomolecular lipid layer, and forming the biomolecular lipid layer characteristic of liposomes. The lipid encapsulated compositions of the present invention can either be a mixture of a variety of types and sizes of liposomes and lipid vesicles or compositions containing one or more predominant types and sizes. A variety of methods are available for separating the liposomes formed into groups based on size and type. These include, for instance, separation using sedimentation, gel filtration, or extrusion through straight pored filters. To make compositions that contain a mixture of liposomal types or sizes, after liposome formation, procedures for separation of the liposome into size/type groups are not employed.

Administration by the sublingual or buccal avoids the first-pass effect from liver metabolism and exposure to the intestines. The compositions of the present invention may be in the oral cavity for a period of time sufficient for absorption sublingually.

The vasoactive amino acid ester compounds and the nutraceutical effective to enhance sexual performance encapsulated in a liposome may be provided together in a tablet for according to methods well known in the art.

Tablets may be defined as the solid unit dosage form of medicament or medicaments with or without suitable diluents and prepared either by molding or by compression. It comprises a mixture of active substances and excipients, usually in powder form, pressed or compacted from a powder into a solid dose. The excipients can include diluents, binders or granulating agents, glidants (flow aids) and lubricants to ensure efficient tabletting; disintegrants to promote tablet break-up in the digestive tract; sweeteners or flavours to enhance taste; and pigments to make the tablets visually attractive. A polymer coating is often applied to make the tablet smoother and easier to swallow, to control the release rate of the active ingredient, to make it more resistant to the environment (extending its shelf life), or to enhance the tablet's appearance.

A tablet can be formulated to deliver an accurate dosage to a specific site; it is usually taken orally, but can be administered orally, sublingually, buccally, rectally or intravaginally.

Most formulations include excipients. Normally, a pharmacologically inactive ingredient (excipient) termed a binder is added to help hold the tablet together and give it strength. A wide variety of binders may be used, some common ones including lactose, dibasic calcium phosphate, sucrose, corn (maize) starch, microcrystalline cellulose, povidone polyvinylpyrrolidone and modified cellulose (for example hydroxypropyl methylcellulose and hydroxyethylcellulose).

The process for making tablets usually include that the active ingredient and excipients are weighed and mixed, the wet granulate is prepared by adding the liquid binder-adhesive to the powder blend and mixing thoroughly (examples of binders/adhesives include aqueous preparations of cornstarch, natural gums such as acacia, cellulose derivatives such as methyl cellulose, gelatin, and povidone), the damp mass is screened through a mesh to form pellets or granules, the granulation is dried (a conventional tray-dryer or fluid-bed dryer are most commonly used), and the dried granules are passed through a screen of smaller size than the one used for the wet mass to create granules of uniform size. Low shear wet granulation processes use very simple mixing equipment, and can take a considerable time to achieve a uniformly mixed state. High shear wet granulation processes use equipment that mixes the powder and liquid at a very fast rate, and thus speeds up the manufacturing process. Fluid bed granulation is a multiple-step wet granulation process performed in the same vessel to pre-heat, granulate, and dry the powders. It is used because it allows close control of the granulation process.

The embodiments and examples presented herein are illustrative of the general nature of the subject matter claimed and are not limiting. It will be understood by those skilled in the art how these embodiments can be readily modified and/or adapted for various applications and in various ways without departing from the spirit and scope of the subject matter disclosed claimed. The claims hereof are to be understood to include without limitation all alternative embodiments and equivalents of the subject matter hereof Phrases, words and terms employed herein are illustrative and are not limiting. All references cited herein are incorporated by reference in their entirety.

1. A composition for enhancing or improving sexual performance or for treating sexual dysfunction or impotence comprising:
an effective amount of a compound of formula (I): 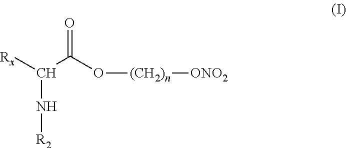
wherein n=1 to 10;
wherein R₁ is an amino acid side chain group (D or L configuration), or derivatives thereof,
wherein R₂ is a hydrogen atom, or an amino acid (D or L configuration) and derivatives thereof, forming a peptide bond;
or any pharmaceutically acceptable salts thereof; and
at least one a nutraceutical effective to enhance sexual performance
in association with a pharmaceutically acceptable carrier.

2. The composition of claim 1, wherein said compound of formula (I) is (2-nitrooxy)-2-ethylamino-3-methylbutanoate: 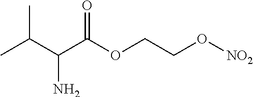
or any pharmaceutically acceptable salts thereof.

3. The composition of claim 1, wherein said compound of formula (I) is valine butylene glycol nitrate: 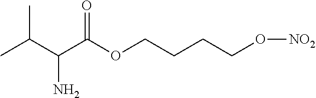 [SMILES: CC(C)C(N)C(=O)OCCCCO[N+](=O)[O-]]
or any pharmaceutically acceptable salts thereof.

4. The composition of claim 1, wherein said compound of formula (I) is 2′-nitrooxy ethyl 2-amino-pentanoate: 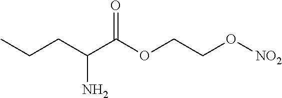
or any pharmaceutically acceptable salts thereof.

5. The composition of claim 1, wherein said compound of formula (I) is 2′-nitrooxy butyl 2-amino-pentanoate: 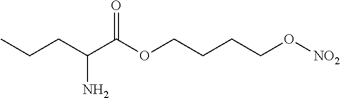 [SMILES: CCCC(N)C(=O)OCCCCO[N+](=O)[O-]]
or any pharmaceutically acceptable salts thereof.

6. The composition of claim 1, wherein R₁ is selected from the group consisting of:

| Originating | | |
|---|---|---|
| Amino acid | Formula | R₁ |
| Glycine | H | H |
| Alanine | CH₃ | CH₃ |
| Valine | (CH₃)₂ | 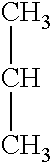 |
| Leucine | CH₂CH(CH₃)₂ | 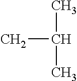 |
| Isoleucine | CH(CH₃)CH₂CH₃ | 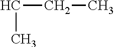 |
| Phenylalanine | CH₂C₆H₅ | 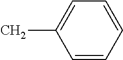 |
| Tyrosine | CH₂C₆H₄OH | 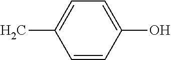 |
| Tryptophane | C₉H₈N | 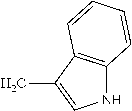 |
| Serine | CH₂OH | H₂C—OH |
| Threonine | CH(OH)CH₃ | 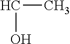 |
| Cysteine | CH₂SH | H₂C—SH |
| Methionine | CH₂CH₂SCH₃ | 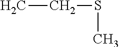 |
| Proline | C₅H₉NO₂ | 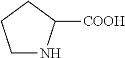 |
| Asparagine | CH₂COCH₂ | 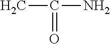 |
| Glutamine | CH₂CH₂CONH₂ | 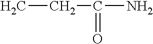 |
| Aspartic acid | CH₂COOH | CH₂—COOH |
| Glutamic acid | CH₂CH₂COOH | H₂C—CH₂—COOH |
| Lysine | CH₂CH₂CH₂CH₂NH₂ | 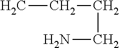 |
| Histidine | CH₃C₃N₂H₃ | 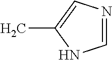 |
| Arginine | (CH₂)₃CN₃H₄ | 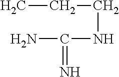 |
| Cystine | CH₂S₂CH₂CHNH₂COOH | 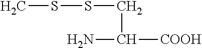 |
| Hydroxyproline | | 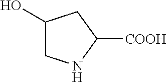 |
| ε-N-methyllysine | CH₂CH₂CH₂CH₂NHCH₃ | 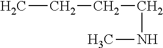 |
| β-alanine | NH₂CH₂CH₂COOH | 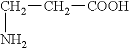 |
| diiodotyrosine | CH₂C₆H₂I₂OH | 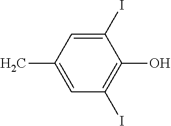 |
| homocysteine | CH₂CH₂SH | H₂C—CH₂—SH |
| ornithine | CH₂CH₂CH₂NH₂ | 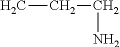 |
| Norvaline | CH₂—CH₃ | CH₂—CH₃ |
| selenocysteine | SeH | SeH |
| Hypusine | CH₂CH₂CH₂CH₂NHCH₂CH(OH)CH₂CH₂NH₂ | 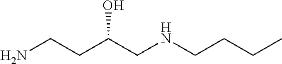 |
| Dehydroalanine | CH₂ | 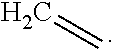 |

7. The composition of claim 1, wherein said R₂ is an amino acid of formula (II) (D or L configuration) and derivatives thereof, forming a peptide bond: 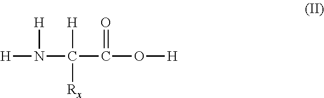
wherein Rₓ is chosen from

| Originating | | |
|---|---|---|
| Amino acid | Formula | Rₓ |
| Glycine | H | H |
| Alanine | CH₃ | CH₃ |
| Valine | (CH₃)₂ | 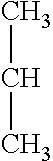 |
| Leucine | CH₂CH(CH₃)₂ | 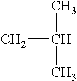 |
| Isoleucine | CH(CH₃)CH₂CH₃ | 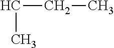 |
| Phenylalanine | CH₂C₆H₅ | 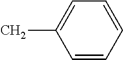 |
| Tyrosine | CH₂C₆H₄OH | 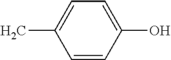 |
| Tryptophane | C₉H₈N | 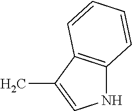 |
| Serine | CH₂OH | H₂C—OH |
| Threonine | CH(OH)CH₃ | 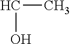 |
| Cysteine | CH₂SH | H₂C—SH |
| Methionine | CH₂CH₂SCH₃ | 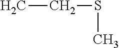 |
| Proline | C₅H₉NO₂ | 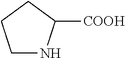 |
| Asparagine | CH₂COCH₂ | 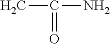 |
| Glutamine | CH₂CH₂CONH₂ | 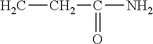 |
| Aspartic acid | CH₂COOH | CH₂—COOH |
| Glutamic acid | CH₂CH₂COOH | H₂C—CH₂—COOH |
| Lysine | CH₂CH₂CH₂CH₂NH₂ | 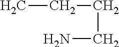 |
| Histidine | CH₃C₃N₂H₃ | 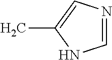 |
| Arginine | (CH₂)₃CN₃H₄ | 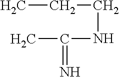 |
| Cystine | CH₂S₂CH₂CHNH₂COOH | 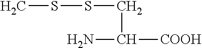 |
| Hydroxyproline | | 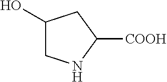 |
| ε-N-methyllysine | CH₂CH₂CH₂CH₂NHCH₃ | 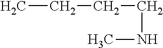 |
| β-alanine | NH₂CH₂CH₂COOH | 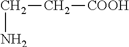 |
| diiodotyrosine | CH₂C₆H₂I₂OH | 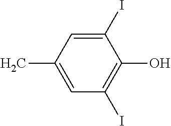 |
| homocysteine | CH₂CH₂SH | H₂C—CH₂—SH |
| ornithine | CH₂CH₂CH₂NH₂ | 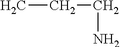 |
| Norvaline | CH₂—CH₃ | CH₂—CH₃ |
| selenocysteine | SeH | SeH |
| Hypusine | CH₂CH₂CH₂CH₂NHCH₂CH(OH)CH₂CH₂NH₂ | 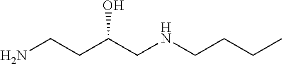 |
| Dehydroalanine | CH₂ | 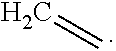 |

8. The composition of claim 1, wherein n=2.

9. The composition of claim 1, wherein the nutraceutical effective to enhance sexual performance is selected from the group consisting of Pausinystalia yohimbe, yohimbine, alpha-yohimbine, and muira puama.

10. The composition of claim 1 wherein the nutraceutical effective to enhance sexual performance is encapsulated in a liposome.

11. A method for enhancing or improving sexual performance or for treating sexual dysfunction or impotence comprising administering an effective amount of:
a compound of formula (I): 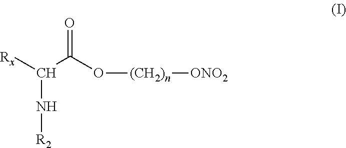
wherein n=1 to 10;
wherein R₁ is an amino acid side chain group (D or L configuration), or derivatives thereof,
wherein R₂ is a hydrogen atom, or an amino acid (D or L configuration) and derivatives thereof, forming a peptide bond;
or any pharmaceutically acceptable salts thereof; and
at least one a nutraceutical effective to enhance sexual performance
in association with a pharmaceutically acceptable carrier.

12. The method of claim 11, wherein said compound of formula (I) is (2-nitrooxy)-2-ethylamino-3-methylbutanoate: 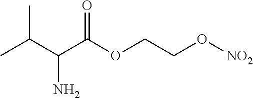
or any pharmaceutically acceptable salts thereof.

13. The method of claim 11, wherein said compound of formula (I) is valine butylene glycol nitrate: 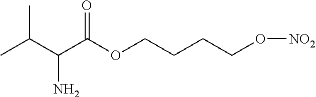 [SMILES: CC(C)C(N)C(=O)OCCCCO[N+](=O)[O-]]
or any pharmaceutically acceptable salts thereof.

14. The method of claim 11, wherein the compound of formula (I) is 2′-nitrooxy ethyl 2-amino-pentanoate: 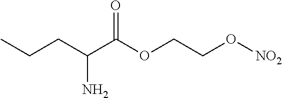
or any pharmaceutically acceptable salts thereof.

15. The method of claim 11 wherein the compound of formula (I) is 2′-nitrooxy butyl 2-amino-pentanoate: 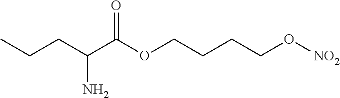 [SMILES: CCCC(N)C(=O)OCCCCO[N+](=O)[O-]]
or any pharmaceutically acceptable salts thereof.

16. The method of claim 11, wherein R₁ is selected from the group consisting of:
Originating

| Amino acid | Formula | R₁ |
|---|---|---|
| Glycine | H | H |
| Alanine | CH₃ | CH₃ |
| Valine | (CH₃)₂ | 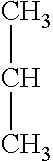 |
| Leucine | CH₂CH(CH₃)₂ | 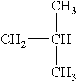 |
| Isoleucine | CH(CH₃)CH₂CH₃ | 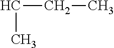 |
| Phenylalanine | CH₂C₆H₅ | 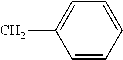 |
| Tyrosine | CH₂C₆H₄OH | 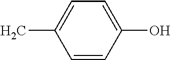 |
| Tryptophane | C₉H₈N | 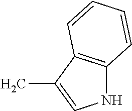 |
| Serine | CH₂OH | H₂C—OH |
| Threonine | CH(OH)CH₃ | 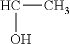 |
| Cysteine | CH₂SH | H₂C—SH |
| Methionine | CH₂CH₂SCH₃ | 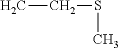 |
| Proline | C₅H₉NO₂ | 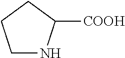 |
| Asparagine | CH₂COCH₂ | 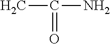 |
| Glutamine | CH₂CH₂CONH₂ | 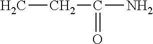 |
| Aspartic acid | CH₂COOH | CH₂—COOH |
| Glutamic acid | CH₂CH₂COOH | H₂C—CH₂—COOH |
| Lysine | CH₂CH₂CH₂CH₂NH₂ | 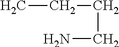 |
| Histidine | CH₃C₃N₂H₃ | 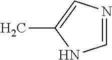 |
| Arginine | (CH₂)₃CN₃H₄ | 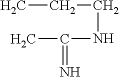 |
| Cystine | CH₂S₂CH₂CHNH₂COOH | 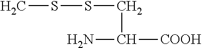 |
| Hydroxyproline | | 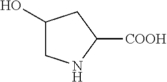 |
| ε-N-methyllysine | CH₂CH₂CH₂CH₂NHCH₃ | 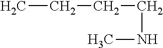 |
| β-alanine | NH₂CH₂CH₂COOH | 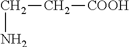 |
| diiodotyrosine | CH₂C₆H₂I₂OH | 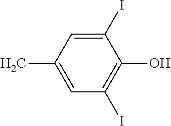 |
| homocysteine | CH₂CH₂SH | H₂C—CH₂—SH |
| ornithine | CH₂CH₂CH₂NH₂ | 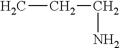 |
| Norvaline | CH₂—CH₃ | CH₂—CH₃ |
| selenocysteine | SeH | SeH |
| Hypusine | CH₂CH₂CH₂CH₂NHCH₂CH(OH)CH₂CH₂NH₂ | 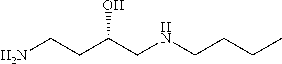 |
| Dehydroalanine | CH₂ | 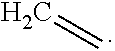 |

17. The method of claim 11 wherein said R₂ is an amino acid of formula (II) (D or L configuration) and derivatives thereof, forming a peptide bond: 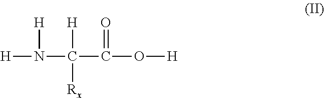
wherein Rₓ is chosen from

| Originating | | |
|---|---|---|
| Amino acid | Formula | Rₓ |
| Glycine | H | H |
| Alanine | CH₃ | CH₃ |
| Valine | (CH₃)₂ | 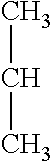 |
| Leucine | CH₂CH(CH₃)₂ | 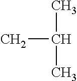 |
| Isoleucine | CH(CH₃)CH₂CH₃ | 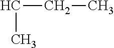 |
| Phenylalanine | CH₂C₆H₅ | 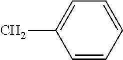 |
| Tyrosine | CH₂C₆H₄OH | 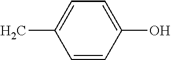 |
| Tryptophane | C₉H₈N | 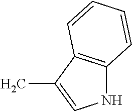 |
| Serine | CH₂OH | H₂C—OH |
| Threonine | CH(OH)CH₃ | 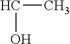 |
| Cysteine | CH₂SH | H₂C—SH |
| Methionine | CH₂CH₂SCH₃ | 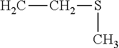 |
| Proline | C₅H₉NO₂ | 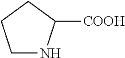 |
| Asparagine | CH₂COCH₂ | 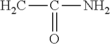 |
| Glutamine | CH₂CH₂CONH₂ | 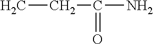 |
| Aspartie acid | CH₂COOH | CH₂—COOH |
| Glutamic acid | CH₂CH₂COOH | H₂C—CH₂—COOH |
| Lysine | CH₂CH₂CH₂CH₂NH₂ | 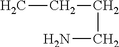 |
| Histidine | CH₃C₃N₂H₃ | 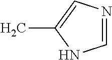 |
| Arginine | (CH₂)₃CN₃H₄ | 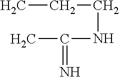 |
| Cystine | CH₂S₂CH₂CHNH₂COOH | 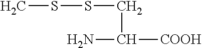 |
| Hydroxyproline | | 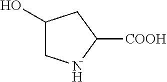 |
| ε-N-methyllysine | CH₂CH₂CH₂CH₂NHCH₃ | 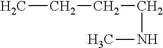 |
| β-alanine | NH₂CH₂CH₂COOH | 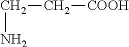 |
| diiodotyrosine | CH₂C₆H₂I₂OH | 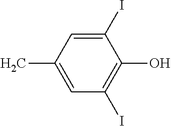 |
| homocysteine | CH₂CH₂SH | H₂C—CH₂—SH |
| ornithine | CH₂CH₂CH₂NH₂ | 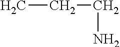 |
| Norvaline | CH₂—CH₃ | CH₂—CH₃ |
| selenocysteine | SeH | SeH |
| Hypusine | CH₂CH₂CH₂CH₂NHCH₂CH(OH)CH₂CH₂NH₂ | 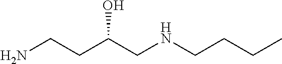 |
| Dehydroalanine | CH₂ | 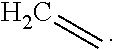 |

18. The method of claim 11 wherein n=2.

19. The method of claim 11 wherein the nutraceutical effective to enhance sexual performance is selected from the group consisting of Pausinystalia yohimbe, yohimbine, alpha-yohimbine, and muira puama.

20. The method of claim 11 wherein the nutraceutical effective to enhance sexual performance is encapsulated in a liposome.

21. The method of claim 11 wherein the composition is in tablet form.

22. The method of claim 11 wherein the composition is administered sublingually.
